(19) United States
(12) Patent Application Publication
(10) Pub. No.: US 2025/0288203 A1
(43) Pub. Date: Sep. 18, 2025

(54) CONTACTLESS TONOMETER AND MEASUREMENT TECHNIQUES FOR USE WITH SURGICAL TOOLS

(71) Applicant: ForSight Robotics Ltd., Yokneam Illit (IL)

(72) Inventors: Ori Ganoni, Ramat Ishay (IL); Daniel Glozman, Kfar Yona (IL)

(21) Appl. No.: 18/860,829

(22) PCT Filed: Apr. 25, 2023
(86) PCT No.: PCT/IB2023/054217

§ 371 Date: Oct. 28, 2024

Related U.S. Application Data
(60) Provisional application No. 63/335,751, filed on Apr. 28, 2022.

(51) Int. Cl. A61B 3/16; A61B 3/14; A61B 5/00

(52) U.S. Cl. CPC A61B 3/165; A61B 3/14; A61B 5/746

(57) ABSTRACT

Apparatus and methods are described including at least one light source (22) configured to direct light toward an eye of a patient. Two or more cameras (28) detect light from the light source that is reflected from a surface (24) of the patient's eye (26). A computer processor (30) receives data from the two or more cameras (28), derives the curvature of the surface (24) of the patient's eye (26) from the received data, and derives intraocular pressure of the patient's eye (26) and/or a change in intraocular pressure of the patient's eye (26) from the derived curvature. Other applications are also described.

### CROSS-REFERENCES TO RELATED APPLICATIONS

The present application claims priority from U.S. Provisional Patent Application No. 63/335,751 to Glozman, filed Apr. 28, 2022, entitled “Measurement techniques for use with surgical tools,” which is incorporated herein by reference.

### FIELD OF EMBODIMENTS OF THE INVENTION

Some applications of the present invention generally relate to medical apparatus and methods. Specifically, some applications of the present invention relate to apparatus and methods for performing measurements on a patient's body.

### BACKGROUND

Cataract surgery involves the removal of the natural lens of the eye that has developed an opacification (known as a cataract), and its replacement with an intraocular lens. Such surgery typically involves a number of standard steps, which are performed sequentially.

In an initial step, the patient's face around the eye is disinfected (typically, with iodine solution), and their face is covered by a sterile drape, such that only the eye is exposed. When the disinfection and draping has been completed, the eye is anesthetized, typically using a local anesthetic, which is administered in the form of liquid eye drops. The eyeball is then exposed, using an eyelid speculum that holds the upper and lower eyelids open. One or more incisions (and typically two or three incisions) are made in the cornea of the eye. The incision(s) are typically made using a specialized blade, which is called a keratome blade. At this stage, lidocaine is typically injected into the anterior chamber of the eye, in order to further anesthetize the eye. Following this step, a viscoelastic injection is applied via the corneal incision(s). The viscoelastic injection is performed in order to stabilize the anterior chamber and to help maintain eye pressure during the remainder of the procedure, and also in order to distend the lens capsule.

In a subsequent stage, known as capsulorhexis, a part of the anterior lens capsule is removed. Various enhanced techniques have been developed for performing capsulorhexis, such as laser-assisted capsulorhexis, zepto-rhexis (which utilizes precision nano-pulse technology), and marker-assisted capsulorhexis (in which the cornea is marked using a predefined marker, in order to indicate the desired size for the capsule opening).

Subsequently, it is common for a fluid wave to be injected via the corneal incision, in order to dissect the cataract's outer cortical layer, in a step known as hydrodissection. In a subsequent step, known as hydrodelineation, the outer softer epi-nucleus of the lens is separated from the inner firmer endo-nucleus by the injection of a fluid wave. In the next step, ultrasonic emulsification of the lens is performed, in a process known as phacoemulsification. The nucleus of the lens is broken initially using a chopper, following which the outer fragments of the lens are broken and removed, typically using an ultrasonic phacoemulsification probe. Further typically, a separate tool is used to perform suction during the phacoemulsification. When the phacoemulsification is complete, the remaining lens cortex (i.e., the outer layer of the lens) material is aspirated from the capsule. During the phacoemulsification and the aspiration, aspirated fluids are typically replaced with irrigation of a balanced salt solution, in order to maintain fluid pressure in the anterior chamber. In some cases, if deemed to be necessary, then the capsule is polished. Subsequently, the intraocular lens (IOL) is inserted into the capsule. The IOL is typically foldable and is inserted in a folded configuration, before unfolding inside the capsule. At this stage, the viscoelastic is removed, typically using the suction device that was previously used to aspirate fluids from the capsule. If necessary, the incision(s) is sealed by elevating the pressure inside the bulbus oculi (i.e., the globe of the eye), causing the internal tissue to be pressed against the external tissue of the incision, such as to force closed the incision.

### SUMMARY

In accordance with some applications of the present invention, a light source, which is typically a point light source such as a laser and/or a LED, directs light toward a patient's eye. Typically, the light is reflected from a surface of the patient's eye, such as the patient's conjunctiva or sclera. Further typically, two or more cameras are configured to detect light from the light source that is reflected from the surface of the patient's eye. A computer processor receives data from the two or more cameras, derives the curvature of the surface from the received data, and derives the patient's intraocular pressure and/or changes in the patient's intraocular pressure from the derived curvature.

As described above, the computer processor derives the intraocular pressure and/or changes in the patient's intraocular pressure from the curvature of the surface. For some applications, the computer processor derives the patient's intraocular pressure by comparing the curvature of the surface to a baseline curvature; the patient's intraocular pressure having been independently measured at the baseline curvature. It is typically assumed that the curvature of the surface will vary from the baseline curvature as the patient's intraocular pressure changes, in accordance with a predetermined relationship. Therefore, the computer processor derives the patient's intraocular pressure by comparing the curvature of the surface to the baseline curvature and the corresponding intraocular pressure, and using the predetermined relationship. For some applications, the computer processor derives the patient's intraocular pressure from the curvature of the surface without reference to a baseline curvature and/or intraocular pressure. For example, the computer processor may use a predetermined relationship between intraocular pressure and curvature of the surface for the general population, for a particular cohort to which the patient belongs, and/or for the patient herself/himself. Alternatively or additionally, the computer processor derives a change in the patient's intraocular pressure from the curvature of the surface without reference to a baseline curvature and/or intraocular pressure. For example, the computer processor may measure the curvature of the surface at the beginning of an ophthalmic procedure. At a given stage in the procedure, or at the end of the procedure, the computer processor again measures the curvature of the surface in order to determine whether intraocular pressure has not changed substantially from the patient's intraocular pressure at the beginning of the procedure.

For some applications, a laser is coupled to an end effector of a robotic unit and is configured to emit a laser beam from the end effector, in accordance with some applications of the present invention. Typically, the robotic unit is used to perform surgery (e.g., microsurgery) with respect to a portion of the patient's body, such as general surgery, ophthalmic surgery, orthopedic surgery, gynecological surgery, otolaryngology, neurosurgery, oral and maxillofacial surgery, plastic surgery, podiatric surgery, vascular surgery, and/or pediatric surgery. Typically, during the surgical procedure, a single camera and/or a stereoscopic camera rig acquires images of the portion of the patient's body upon which the procedure is being performed and/or of the tool that is mounted within the end effector. For some applications, a computer processor derives information regarding the disposition (i.e., location and/or orientation) of the tool relative to the portion of the patient's body by analyzing the images. Typically, in response to the derived information regarding the disposition of the tool relative to the portion of the patient's body, the computer processor drives the robotic unit to move the tool and/or to actuate the tool to perform a given function.

Typically, when a single camera is used, the computer processor identifies a point at which the laser beam intersects with a surface of the patient's body, and thereby derives information regarding the position and orientation of the tool relative to the portion of the patient's body. Further typically, when a stereoscopic camera rig is used, the computer processor triangulates the data from the two cameras in order to derive the orientation of the laser beam, and thereby derives information regarding the position and orientation of the tool relative to the portion of the patient's body. It is noted that in order to derive information regarding the position of the tool based on upon the point at which the laser beam intersects with the surface of the patient's body or based upon the orientation of the laser beam, it is assumed that the laser and the tool are disposed on the end effector at known respective positions, such that the position of the laser with respect to the tool is known.

For some applications, the computer processor identifies the tool and the portion of the patient's body within the images and thereby derives the position and orientation of the tool relative to the portion of the patient's body, as described above. For some such applications, the computer processor then validates the derived position and orientation of the tool relative to the portion of the patient's body by identifying the laser beam and/or a portion thereof.

There is therefore provided, in accordance with some applications of the present invention, apparatus including:
at least one light source configured to direct light toward an eye of a patient;
two or more cameras configured to detect light from the light source that is reflected from a surface of the patient's eye; and
at least one computer processor configured to:
   receive data from the two or more cameras,
   derive a curvature of the surface of the patient's eye from the received data, and
   derive intraocular pressure of the patient's eye and/or a change in intraocular pressure of the patient's eye from the derived curvature.

In some applications, the computer processor is configured to receive an indication of a baseline intraocular pressure of the patient's eye and a corresponding baseline curvature of the surface of the patient's eye, and the computer processor is configured to derive the intraocular pressure of the patient's eye by comparing the derived curvature to the baseline curvature.

In some applications, the computer processor is configured to derive the intraocular pressure of the patient's eye by using a predetermined relationship between intraocular pressure and curvature of the surface for a set of one or more people.

In some applications, the computer processor is configured to determine whether the patient's intraocular pressure has changed with respect to the patient's intraocular pressure at a previous time by comparing the derived curvature to a curvature of the surface that was measured at the previous time.

In some applications, the computer processor is configured to determine whether the patient's intraocular pressure has changed with respect to the patient's intraocular pressure at the beginning of an ophthalmic procedure by comparing the derived curvature to a curvature of the surface that was measured at the beginning of the ophthalmic procedure.

In some applications, the computer processor is configured to determine whether the patient's intraocular pressure has changed with respect to the patient's intraocular pressure at the beginning of a cataract surgery by comparing the derived curvature to a curvature of the surface that was measured at the beginning of the cataract surgery.

In some applications, the apparatus further includes an output device, and the computer processor is configured to generate an output on the output device in response to the derived intraocular pressure of the patient's eye and/or the derived change in intraocular pressure of the patient's eye.

In some applications, the computer processor is configured to generate an alert in response to the derived change in intraocular pressure being greater than a given threshold.

In some applications, the computer processor is configured to generate an alert in response to the derived intraocular pressure being greater than a given threshold.

In some applications, the computer processor is configured to generate an alert in response to the derived intraocular pressure being below a given threshold.

In some applications, the apparatus further includes a robotic unit, and the computer processor is configured to control the robotic unit in response to the derived intraocular pressure of the patient's eye and/or the derived change in intraocular pressure of the patient's eye.

In some applications, the computer processor is configured to prevent the robotic unit from performing a stage of a procedure in response to the derived change in intraocular pressure being greater than a given threshold.

In some applications, the computer processor is configured to prevent the robotic unit from performing a stage of a procedure in response to the derived intraocular pressure being greater than a given threshold.

In some applications, the computer processor is configured to prevent the robotic unit from performing a stage of a procedure in response to the derived intraocular pressure being below a given threshold.

There is therefore provided, in accordance with some applications of the present invention, a method including:
direct light toward an eye of a patient;
detect light that is reflected from a surface of the patient's eye, using at least one camera; and
using at least one computer processor:
   receiving data from the two or more cameras;
   deriving a curvature of the surface of the patient's eye from the received data; and
   deriving intraocular pressure of the patient's eye and/or a change in intraocular pressure of the patient's eye from the derived curvature.

In some applications, the method further includes driving the computer processor to receive an indication of a baseline intraocular pressure of the patient's eye and a corresponding baseline curvature of the surface of the patient's eye, and deriving the intraocular pressure of the patient's eye includes comparing the derived curvature to the baseline curvature.

In some applications, deriving the intraocular pressure of the patient's eye includes using a predetermined relationship between intraocular pressure and curvature of the surface for a set of one or more people.

In some applications, the method further includes determining whether the patient's intraocular pressure has changed with respect to the patient's intraocular pressure at a previous time by comparing the derived curvature to a curvature of the surface that was measured at the previous time.

In some applications, determining whether the patient's intraocular pressure has changed with respect to the patient's intraocular pressure at a previous time includes determining whether the patient's intraocular pressure has changed with respect to the patient's intraocular pressure at the beginning of an ophthalmic procedure by comparing the derived curvature to a curvature of the surface that was measured at the beginning of the ophthalmic procedure.

In some applications, determining whether the patient's intraocular pressure has changed with respect to the patient's intraocular pressure at the beginning of an ophthalmic procedure includes determining whether the patient's intraocular pressure has changed with respect to the patient's intraocular pressure at the beginning of a cataract surgery by comparing the derived curvature to a curvature of the surface that was measured at the beginning of the cataract surgery.

In some applications, the method further includes generating an output on an output device in response to the derived intraocular pressure of the patient's eye and/or the derived change in intraocular pressure of the patient's eye.

In some applications, generating the output on the output device includes generating an alert in response to the derived change in intraocular pressure being greater than a given threshold.

In some applications, generating the output on the output device includes generating an alert in response to the derived intraocular pressure being greater than a given threshold.

In some applications, generating the output on the output device includes generating an alert in response to the derived intraocular pressure being below a given threshold.

In some applications, the method further includes controlling a robotic unit in response to the derived intraocular pressure of the patient's eye and/or the derived change in intraocular pressure of the patient's eye.

In some applications, controlling the robotic unit includes preventing the robotic unit from performing a stage of a procedure in response to the derived change in intraocular pressure being greater than a given threshold.

In some applications, controlling the robotic unit includes preventing the robotic unit from performing a stage of a procedure in response to the derived intraocular pressure being greater than a given threshold

In some applications, controlling the robotic unit includes preventing the robotic unit from performing a stage of a procedure in response to the derived intraocular pressure being below a given threshold.

There is further provided, in accordance with some applications of the present invention, apparatus including:
a surgical instrument configured to perform a procedure on a portion of a body of patient;
at least one laser coupled to the surgical instrument and configured to emit a laser beam toward the portion of the patient's body;
one or more cameras configured to image the portion of the patient's body; and
at least one computer processor configured to:
   receive data from the one or more cameras,
   identify a disposition of at least a portion of the laser beam with respect to the portion of the patient's body based on the received data, and
   derive information regarding a disposition of the surgical instrument with respect to the portion of the patient's body from the identified disposition of the laser beam with respect to the portion of the patient's body.

In some applications, the computer processor is configured to move the surgical instrument in response to the derived information regarding the disposition of the surgical instrument with respect to the portion of the patient's body.

In some applications, the computer processor is configured to actuate the surgical instrument to perform a function in response to the derived information regarding the disposition of the surgical instrument with respect to the portion of the patient's body.

In some applications, the computer processor is configured to receive data from a single camera of the one or more cameras and the computer processor is configured to identify a disposition of at least the portion of the laser beam with respect to the portion of the patient's body based on the received data by identifying a point at which the laser beam intersects with a surface of the patient's body.

In some applications, the computer processor is configured to receive data from a stereoscopic camera rig including two or more cameras and wherein the computer processor is configured to identify a disposition of at least the portion of the laser beam with respect to the portion of the patient's body based on the received data by triangulating the data from the two or more cameras in order to derive the disposition of the laser beam.

In some applications, the at least one laser includes two or more lasers configured to emit respective laser beams, with each of the lasers having a respective, different orientation with respect to the surgical instrument, and the computer processor triangulates the laser beams to derive information regarding the disposition of the surgical instrument with respect to the portion of the patient's body.

In some applications, the computer processor is configured to:
receive images of the surgical instrument and the portion of the patient's body from the one or more camera;
identify the surgical instrument and the portion of the patient's body within the images; and
thereby derive an initial derived disposition of the surgical instrument relative to the portion of the patient's body.

In some applications, the computer processor is configured to validate the initial derived disposition of the surgical instrument relative to the portion of the patient's body using the identified disposition of the laser beam with respect to the portion of the patient's body.

In some applications, the computer processor is configured to refine the initial derived disposition of the surgical instrument relative to the portion of the patient's body using the identified disposition of the portion of the laser beam with respect to the portion of the patient's body.

In some applications, over a course of a procedure, the computer processor is configured to periodically derive an initial derived disposition of the surgical instrument relative to the portion of the patient's body and to refine the initial derived disposition of the surgical instrument relative to the portion of the patient's body using the identified disposition of the portion of the laser beam with respect to the portion of the patient's body.

In some applications, the surgical instrument includes a tool mounted in an end effector of a robotic arm.

In some applications, the laser is coupled to the end effector.

In some applications, the laser is coupled to the tool.

In some applications, the robotic unit is configured to perform ophthalmic surgery on the patient.

In some applications, the laser is configured to generate a pattern of light, flashes of light, and/or different colors of light in order to convey additional information to the computer processor.

In some applications:
the surgical instrument includes a tool mounted in an end effector of a robotic arm,
the laser is coupled to the end effector, and
the computer processor is configured to derive which tool is currently mounted on the end effector, based on the color, the length of the flashes, and/or the pattern of laser light.

There is further provided, in accordance with some applications of the present invention, a method including:
performing a procedure on a portion of a body of patient using a surgical instrument;
driving at least one laser coupled to the surgical instrument to emit a laser beam toward the portion of the patient's body;
driving one or more cameras configured to image the portion of the patient's body; and
driving at least one computer processor to:
   receive data from the one or more cameras,
   identify a disposition of at least a portion of the laser beam with respect to the portion of the patient's body based on the received data, and
   derive information regarding a disposition of the surgical instrument with respect to the portion of the patient's body from the identified disposition of the laser beam with respect to the portion of the patient's body.

In some applications, performing the procedure on the portion of the patient's body includes performing ophthalmic surgery on the patient.

In some applications, the method further includes driving the computer processor to move the surgical instrument in response to the derived information regarding the disposition of the surgical instrument with respect to the portion of the patient's body.

In some applications, the method further includes driving the computer processor to actuate the surgical instrument to perform a function in response to the derived information regarding the disposition of the surgical instrument with respect to the portion of the patient's body.

In some applications, the computer processor is configured to receive data from a single camera of the one or more cameras and the computer processor is configured to identify a disposition of at least the portion of the laser beam with respect to the portion of the patient's body based on the received data by identifying a point at which the laser beam intersects with a surface of the patient's body.

In some applications, the computer processor is configured to receive data from a stereoscopic camera rig including two or more cameras and the computer processor is configured to identify a disposition of at least the portion of the laser beam with respect to the portion of the patient's body based on the received data by triangulating the data from the two or more cameras in order to derive the disposition of the laser beam.

In some applications, the at least one laser includes two or more lasers configured to emit respective laser beams, with each of the lasers having a respective, different orientation with respect to the surgical instrument, and the computer processor triangulates the laser beams to derive information regarding the disposition of the surgical instrument with respect to the portion of the patient's body.

In some applications, the computer processor is configured to:
receive images of the surgical instrument and the portion of the patient's body from the one or more camera;
identify the surgical instrument and the portion of the patient's body within the images; and
thereby derive an initial derived disposition of the surgical instrument relative to the portion of the patient's body.

In some applications, the computer processor is configured to validate the initial derived disposition of the surgical instrument relative to the portion of the patient's body using the identified disposition of the laser beam with respect to the portion of the patient's body.

In some applications, the computer processor is configured to refine the initial derived disposition of the surgical instrument relative to the portion of the patient's body using the identified disposition of the portion of the laser beam with respect to the portion of the patient's body.

In some applications, over a course of a procedure, the computer processor is configured to periodically derive an initial derived disposition of the surgical instrument relative to the portion of the patient's body and to refine the initial derived disposition of the surgical instrument relative to the portion of the patient's body using the identified disposition of the portion of the laser beam with respect to the portion of the patient's body.

In some applications, the method further includes driving the laser to generate a pattern of light, flashes of light, and/or different colors of light in order to convey additional information to the computer processor.

In some applications:
the surgical instrument includes a tool mounted in an end effector of a robotic arm,
the laser is coupled to the end effector, and
the method includes the computer processor deriving which tool is currently mounted on the end effector, based on the color, the length of the flashes, and/or the pattern of laser light.

In some applications, the surgical instrument includes a tool mounted in an end effector of a robotic arm.

In some applications, the laser is coupled to the end effector.

In some applications, the laser is coupled to the tool.

There is further provided, in accordance with some applications of the present invention, apparatus including:
a tool mounted in an end effector of a robotic arm, the tool being configured to perform a procedure on a portion of a body of patient;
at least one laser configured to generate a pattern of light, flashes of light, and/or different colors of light that is directed toward the portion of the patient's body;
one or more cameras configured to image the portion of the patient's body; and
at least one computer processor configured to:
   receive data from the one or more cameras,
   determine which tool is currently mounted on the end effector, based on the color, the length of the flashes, and/or the pattern of laser light.

There is further provided, in accordance with some applications of the present invention, a method including:
performing a procedure on a portion of a body of patient, using a tool mounted in an end effector of a robotic arm;
generating a pattern of light, flashes of light, and/or different colors of light that is directed toward the portion of the patient's body, using at least one laser;
imaging the portion of the patient's body, using one or more cameras; and
using at least one computer processor:
   receiving data from the one or more cameras; and
   determining which tool is currently mounted on the end effector, based on the color, the length of the flashes, and/or the pattern of laser light.

The present invention will be more fully understood from the following detailed description of embodiments thereof, taken together with the drawings, in which:

### BRIEF DESCRIPTION OF THE DRA WINGS

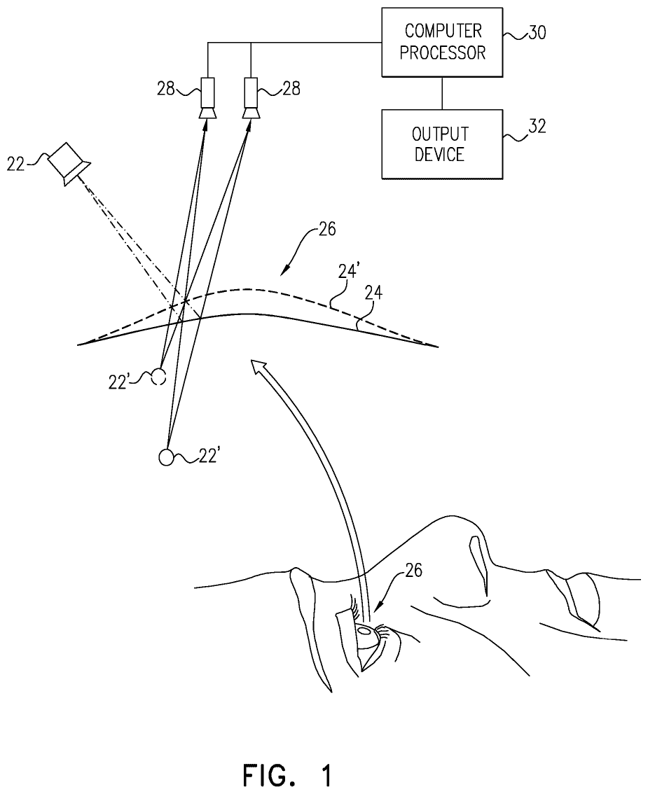

FIG. 1 is a schematic illustration of a system for deriving a patient's intraocular pressure, in accordance with some applications of the present invention;

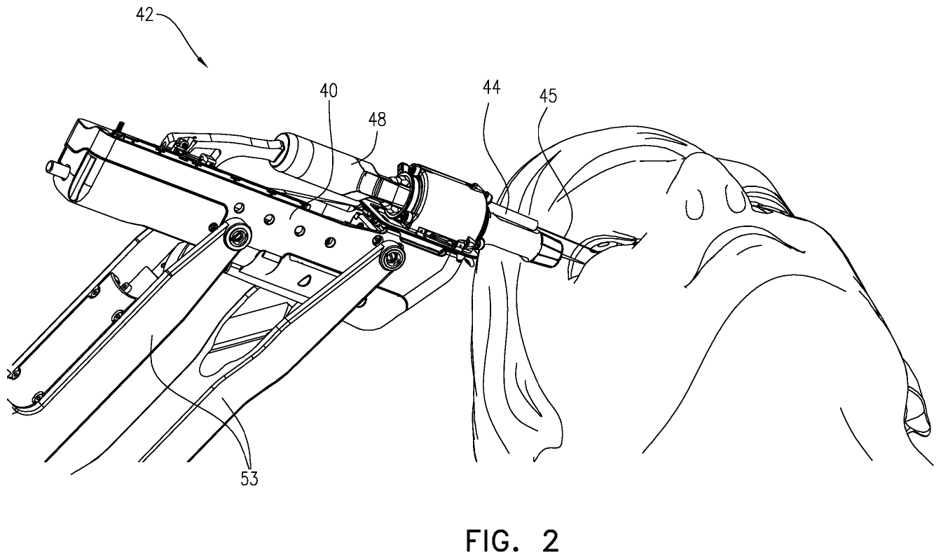

FIG. 2 is a picture of an end effector of a robotic unit with a laser beam being generated from the end effector or from a tool disposed on the end effector, in accordance with some applications of the present invention;

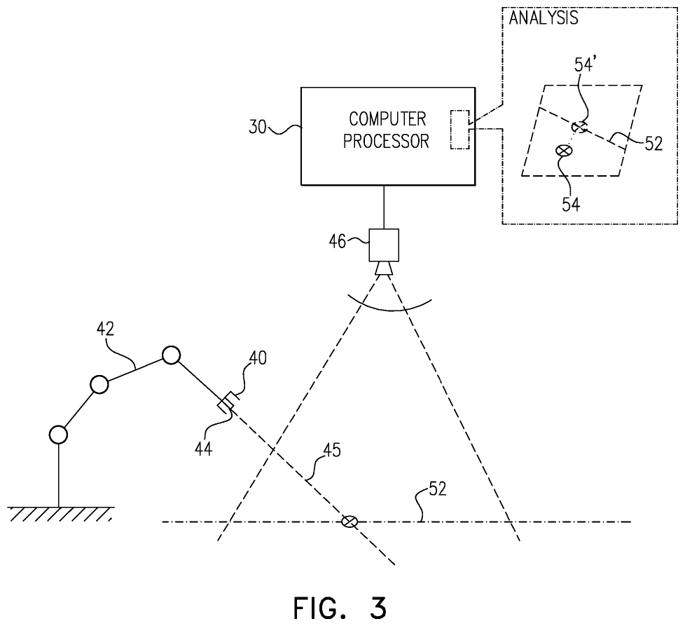

FIG. 3 is a schematic illustration of a single-camera-based system for deriving the information regarding the position of a surgical instrument with respect to the portion of the patient's body, in accordance with some applications of the present invention; and

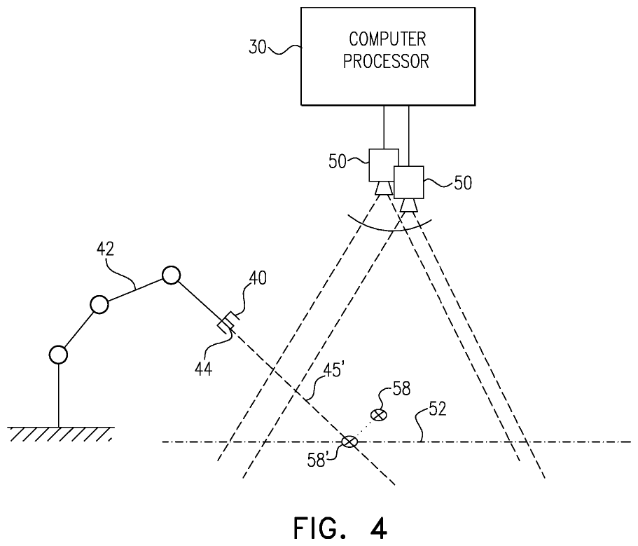

FIG. 4 is a schematic illustration of a stereoscopic-camera-based system for deriving information regarding the position of a surgical instrument with respect to the portion of the patient's body, in accordance with some applications of the present invention.

### DETAILED DESCRIPTION OF EMBODIMENTS

Reference is now made to FIG. 1, which is a schematic illustration of a system for deriving a patient's intraocular pressure, in accordance with some applications of the present invention. As shown in FIG. 1, for some applications, a light source 22, which is typically a point light source such as a laser and/or a LED, directs light toward a patient's eye. Typically, the light is reflected from a surface 24 of the patient's eye 26, such as the patient's conjunctiva or sclera. Further typically, two or more cameras 28 are configured to detect light from the light source that is reflected from surface 24 of the patient's eye. A computer processor 30 receives data from the two or more cameras, derives the curvature of surface 24 from the received data, and derives the patient's intraocular pressure and/or changes in the patient's intraocular pressure from the derived curvature.

As schematically illustrated in FIG. 1, as the curvature of surface 24 undergoes a change e.g., from the shape indicated by the solid curve to the shape indicated by the dashed curve (indicated by reference numeral 24′), the location of a virtual light source 22′ that is generated by the reflected light changes. For some applications, the computer processor determines the curvature of surface 24 based on the location of light source 22 with respect to the patient's eye and the location of the virtual light source that is detected by the cameras. The computer processor derives the intraocular pressure and/or changes in the patient's intraocular pressure from the curvature of surface 24. For some applications, the computer processor derives the patient's intraocular pressure by comparing the curvature of surface 24 to a baseline curvature; the patient's intraocular pressure having been independently measured at the baseline curvature. It is typically assumed that the curvature of surface 24 will vary from the baseline curvature as the patient's intraocular pressure changes, in accordance with a predetermined relationship. Therefore, the computer processor derives the patient's intraocular pressure by comparing the curvature of the surface to the baseline curvature and the corresponding intraocular pressure, and using the predetermined relationship. For some applications, the computer processor derives the patient's intraocular pressure from the curvature of surface 24 without reference to a baseline curvature and/or intraocular pressure. For example, the computer processor may use a predetermined relationship between intraocular pressure and curvature of the surface for a set of one or more people, e.g., the general population, a particular cohort to which the patient belongs, and/or the patient herself/himself. Alternatively or additionally, the computer processor derives a change in the patient's intraocular pressure from the curvature of surface 24 without reference to a baseline curvature and/or intraocular pressure. For example, the computer processor may measure the curvature of surface 24 at the beginning of an ophthalmic procedure (such as cataract surgery). At a given stage in the procedure, or at the end of the procedure, the computer processor again measures the curvature of surface 24 in order to determine whether intraocular pressure has not changed substantially from the patient's intraocular pressure at the beginning of the procedure.

For some applications, generally similar techniques to those described above for deriving a patient's intraocular pressure and/or changes in the patient's intraocular pressure are used. However, the curvature of surface 24 is derived in a different manner to that described above, as an alternative to or in addition to deriving the curvature of surface 24 based on the reflection of light from light source 22. For example, the computer processor may detect two or more features that are located on the surface (e.g., veins, features of the iris, and/or opposing sides of the limbus (i.e., the junction between the sclera and the cornea)) and based on the distance between the features, the computer processor derives the curvature of the surface. For such applications, the computer processor typically then derives the patient's intraocular pressure and/or changes in the patient's intraocular pressure from the curvature of the surface, using one or more of the techniques described hereinabove.

For some applications, computer processor 30 generates an output on an output device 32 based upon the determined intraocular pressure. The output device may include a display and/or a different type of audio or visual output device associated with a robotic surgical system. For some applications, the computer processor generates an alert in response to detecting a change in intraocular pressure and/or in response to detecting intraocular pressure that is greater than a given threshold, and/or in response to detecting intraocular pressure that is below a given threshold. For some applications, the computer processor comprises a portion of a robotic surgical system. For example, the computer processor may be used in conjunction with a robotic unit 42, as shown in FIG. 2. For some such applications, the computer processor controls the robotic unit at least partially based upon the determined intraocular pressure. For example, the computer processor may prevent the robotic unit from performing a stage of a procedure in response to detecting a change in intraocular pressure and/or in response to detecting intraocular pressure that is greater than a given threshold, and/or in response to detecting intraocular pressure that is below than a given threshold.

As noted above, for some applications, the computer processor is configured to identify features of the patient's iris and/or other portions of the patient's eye. For some applications, identification of features of the patient's iris and/or other portions of the patient's eye is used for patient-identification purposes. For example, in cases in which robotic surgical procedure is to be performed on the patient's eye, in a preliminary clinical session, a physician typically plans the surgical procedure based on measurements that are performed upon the patient's eye. The physician typically then inputs details of the patient and/or the planning into the robotic surgical system. For some such cases, prior to performing the robotic surgical procedure, the robotic surgical system verifies that the designated patient is being operated upon by identifying features of the patient's iris and/or other portions of the patient's eye. Reference is now made to FIG. 2, which is a picture of an end effector 40 of a robotic unit 42 that schematically illustrates a laser 44 coupled to the end effector (and/or a tool disposed on the end effector) and projecting a laser beam 45 from the end effector, in accordance with some applications of the present invention. Reference is also made to FIG. 3, which is a schematic illustration of a system that uses a single camera 46 for deriving information regarding the disposition (i.e., location and/or orientation) of a surgical instrument 48 (e.g., a tool that is mounted within end effector 40, as shown in FIG. 2) with respect to a portion of a patient's body (e.g., the patient's eye), in accordance with some applications of the present invention, and to FIG. 4, which is a schematic illustration of a system based on a stereoscopic camera rig 50 (comprising two or more cameras) for deriving information regarding the disposition of a surgical instrument with respect to the portion of the patient's body, in accordance with some applications of the present invention.

Typically, robotic unit 42 is used to perform surgery (e.g., microsurgery) with respect to a portion of the patient's body, such as general surgery, ophthalmic surgery, orthopedic surgery, gynecological surgery, otolaryngology, neurosurgery, oral and maxillofacial surgery, plastic surgery, podiatric surgery, vascular surgery, and/or pediatric surgery. In accordance with respective applications, end effector 40 comprises an end effector of a parallel robotic unit, a serial robotic unit, or a hybrid robotic unit. In the example shown in FIG. 2, the end effector is mounted on two pairs of parallel arms 53. Typically, during the surgical procedure, camera 46 and/or stereoscopic camera rig 50 acquires images of the portion of the patient's body upon which the procedure is being performed and/or of the tool that is mounted within the end effector. For some applications, computer processor 30 (shown in FIGS. 3 and 4, for example) derives information regarding the disposition (i.e., location and/or orientation) of the tool relative to the portion of the patient's body by analyzing the images. Typically, in response to the derived information regarding the disposition of the tool relative to the portion of the patient's body, the computer processor drives the robotic unit to move the tool and/or to actuate the tool to perform a given function.

As described above, computer processor 30 typically derives information regarding the position and orientation of the tool relative to the portion of the patient's body by analyzing images of the portion of the patient's body and/or of the tool that are acquired by either single camera 46 and/or stereoscopic camera rig 50. For some such applications, the computer processor identifies the tool and the portion of the patient's body within the images and thereby derives the position and orientation of the tool relative to the portion of the patient's body. Alternatively or additionally, the computer processor identifies the disposition (i.e., location and/or orientation) of laser beam 45 or a portion thereof with respect to the portion of the patient's body within the images, and derives information regarding the disposition of the tool relative to the portion of the patient's body therefrom. Typically, when a single camera is used (as shown in FIG. 3), the computer processor identifies a point at which the laser beam intersects with a surface 52 of the patient's body, and thereby derives information regarding the position and orientation of the tool relative to the portion of the patient's body. Further typically, when a stereoscopic camera rig is used (as shown in FIG. 4), the computer processor triangulates the data from the two or more cameras in order to derive the position and orientation of the laser beam, and thereby derives information regarding the position and orientation of the tool relative to the portion of the patient's body. It is noted that in order to derive information regarding the position of the tool based on upon the point at which the laser beam intersects with surface 52 of the patient's body or based upon the orientation of the laser beam, it is assumed that the laser and the tool are disposed on the end effector at known respective positions, such that the position of the laser with respect to the tool is known.

For some applications, the computer processor identifies the tool and the portion of the patient's body within the images and thereby derives an initial derived position and orientation of the tool relative to the portion of the patient's body, as described above. For some such applications, the computer processor then validates the initial derived position and orientation of the tool relative to the portion of the patient's body by identifying the laser beam and/or a portion thereof. In some cases, the computer processor refines the initial derived position and orientation of the tool relative to the portion of the patient's body by identifying the laser beam and/or a portion thereof. For example, referring to the “Analysis” frame shown in FIG. 3, based on the initial analysis of the image that is acquired by camera 46, the computer processor estimates that the laser beam will intersect surface 52 at estimated intersection point 54′. However, the computer processor identifies that the actual intersection point 54 is offset from estimated intersection point 54′. Alternatively, referring to FIG. 4, based on the initial analysis of the image that is acquired by camera 46, the computer processor estimates that the laser beam is oriented as indicated by path 45′, such that the laser beam will intersect surface 52 at estimated intersection point 58′. However, the computer processor triangulates the position of the laser beam within the stereoscopic images to determine that the actual path of the laser beam is offset from path 56, such that the actual intersection point 58 of the laser beam with surface 52 is offset (three-dimensionally) from estimated intersection point 58′. For some applications, the computer processor periodically refines (e.g., at fixed intervals over the course of a procedure) the position and orientation of the tool with respect to the portion of the patient's body as derived from the images based upon the identified position of the intersection of the laser beam with surface 52 and/or based upon the identified orientation of the laser beam.

For some applications laser 44 is configured to generate a pattern of light, flashes of light, and/or different colors of light (e.g., by using more than one laser diode) in order to convey additional information to the computer processor via camera 46 or stereoscopic camera rig 50. For example, the laser may be configured to indicate which tool is currently mounted on the end effector, based on the color, the length of the flashes, and/or the pattern of laser light. For some applications, two or more lasers (e.g., three or more lasers) emit respective laser beams, with each of the lasers having a respective, different orientation with respect to the end effector. For some such applications, even using single camera 46, the computer processor triangulates the laser beams to derive the position and/or orientation of the tool with respect to the portion of the patient's body.

It is noted that although some applications of the present disclosure have been described in which a tool that is disposed on the end effector of a robotic unit is used as surgical instrument 48, the scope of the present disclosure includes using the apparatus and methods described with reference to FIGS. 2-4 in combination with any tool that is configured to perform a procedure on a portion of a patient's body.

For some applications, the techniques described herein are used for performing ophthalmic surgery on a patient's eye, such as cataract surgery, collagen crosslinking, endothelial keratoplasty (e.g., DSEK, DMEK, and/or PDEK), DSO (descemet stripping without transplantation), laser assisted keratoplasty, keratoplasty, LASIK/PRK, SMILE, pterygium, ocular surface cancer treatment, secondary IOL placement (sutured, transconjunctival, etc.), iris repair, IOL reposition, IOL exchange, superficial keratectomy, Minimally Invasive Glaucoma Surgery (MIGS), limbal stem cell transplantation, astigmatic keratotomy, Limbal Relaxing Incisions (LRI), amniotic membrane transplantation (AMT), glaucoma surgery (e.g., trabs, tubes, minimally invasive glaucoma surgery), automated lamellar keratoplasty (ALK), anterior vitrectomy, and/or pars plana anterior vitrectomy. Alternatively or additionally, the apparatus and methods described herein are applied to other surgical and/or microsurgical procedures, such as general surgery, orthopedic surgery, gynecological surgery, otolaryngology, neurosurgery, oral and maxillofacial surgery, plastic surgery, podiatric surgery, vascular surgery, and/or pediatric surgery that is performed using microsurgical techniques. For some such applications, camera 46 and/r stereoscopic camera rig 50 includes one or more microscopic imaging units.

Applications of the invention described herein can take the form of a computer program product accessible from a computer-usable or computer-readable medium (e.g., a non-transitory computer-readable medium) providing program code for use by or in connection with a computer or any instruction execution system, such as computer processor 30. For the purpose of this description, a computer-usable or computer readable medium can be any apparatus that can comprise, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Typically, the computer-usable or computer readable medium is a non-transitory computer-usable or computer readable medium.

Examples of a computer-readable medium include a semiconductor or solid-state memory, magnetic tape, a removable computer diskette, a random-access memory (RAM), a read-only memory (ROM), a rigid magnetic disk and an optical disk. Current examples of optical disks include compact disk-read only memory (CD-ROM), compact disk-read/write (CD-R/W), DVD, and a USB drive.

A data processing system suitable for storing and/or executing program code will include at least one processor (e.g., computer processor 30) coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution. The system can read the inventive instructions on the program storage devices and follow these instructions to execute the methodology of the embodiments of the invention.

Network adapters may be coupled to the processor to enable the processor to become coupled to other processors or remote printers or storage devices through intervening private or public networks. Modems, cable modem and Ethernet cards are just a few of the currently available types of network adapters.

Computer program code for carrying out operations of the present invention may be written in any combination of one or more programming languages, including an object-oriented programming language such as Java, Smalltalk, C++ or the like and conventional procedural programming languages, such as the C programming language or similar programming languages.

It will be understood that the algorithms described herein, can be implemented by computer program instructions. These computer program instructions may be provided to a processor of a general-purpose computer, special purpose computer, or other programmable data processing apparatus to produce a machine, such that the instructions, which execute via the processor of the computer (e.g., computer processor 30) or other programmable data processing apparatus, create means for implementing the functions/acts specified in the algorithms described in the present application. These computer program instructions may also be stored in a computer-readable medium (e.g., a non-transitory computer-readable medium) that can direct a computer or other programmable data processing apparatus to function in a particular manner, such that the instructions stored in the computer-readable medium produce an article of manufacture including instruction means which implement the function/act specified in the algorithms. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide processes for implementing the functions/acts specified in the algorithms described in the present application.

Computer processor 30 is typically a hardware device programmed with computer program instructions to produce a special purpose computer. For example, when programmed to perform the algorithms described with reference to the Figures, computer processor 30 typically acts as a special purpose surgical-measurement computer processor. Typically, the operations described herein that are performed by computer processor 30 transform the physical state of a memory, which is a real physical article, to have a different magnetic polarity, electrical charge, or the like depending on the technology of the memory that is used. For some applications, operations that are described as being performed by a computer processor are performed by a plurality of computer processors in combination with each other.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus comprising:
at least one light source configured to direct light toward an eye of a patient;
two or more cameras configured to detect light from the light source that is reflected from a surface of the patient's eye; and
at least one computer processor configured to:
receive data from the two or more cameras,
derive a curvature of the surface of the patient's eye from the received data, and
derive intraocular pressure of the patient's eye and/or a change in intraocular pressure of the patient's eye from the derived curvature.

2. The apparatus according to claim 1, wherein the computer processor is configured to receive an indication of a baseline intraocular pressure of the patient's eye and a corresponding baseline curvature of the surface of the patient's eye, and the computer processor is configured to derive the intraocular pressure of the patient's eye by comparing the derived curvature to the baseline curvature.

3. The apparatus according to claim 1, wherein the computer processor is configured to derive the intraocular pressure of the patient's eye by using a predetermined relationship between intraocular pressure and curvature of the surface for a set of one or more people.

4. The apparatus according to claim 1, wherein the computer processor is configured to determine whether the patient's intraocular pressure has changed with respect to the patient's intraocular pressure at a previous time by comparing the derived curvature to a curvature of the surface that was measured at the previous time.

5. The apparatus according to claim 4, wherein the computer processor is configured to determine whether the patient's intraocular pressure has changed with respect to the patient's intraocular pressure at the beginning of an ophthalmic procedure by comparing the derived curvature to a curvature of the surface that was measured at the beginning of the ophthalmic procedure.

6. The apparatus according to claim 4, wherein the computer processor is configured to determine whether the patient's intraocular pressure has changed with respect to the patient's intraocular pressure at the beginning of a cataract surgery by comparing the derived curvature to a curvature of the surface that was measured at the beginning of the cataract surgery.

7. The apparatus according to claim 1, further comprising an output device, wherein the computer processor is configured to generate an output on the output device in response to the derived intraocular pressure of the patient's eye and/or the derived change in intraocular pressure of the patient's eye.

8. The apparatus according to claim 7, wherein the computer processor is configured to generate an alert in response to the derived change in intraocular pressure being greater than a given threshold.

9. The apparatus according to claim 7, wherein the computer processor is configured to generate an alert in response to the derived intraocular pressure being greater than a given threshold.

10. The apparatus according to claim 7, wherein the computer processor is configured to generate an alert in response to the derived intraocular pressure being below a given threshold.

11. The apparatus according to claim 1, further comprising a robotic unit, wherein the computer processor is configured to control the robotic unit in response to the derived intraocular pressure of the patient's eye and/or the derived change in intraocular pressure of the patient's eye.

12. The apparatus according to claim 11, wherein the computer processor is configured to prevent the robotic unit from performing a stage of a procedure in response to the derived change in intraocular pressure being greater than a given threshold.

13. The apparatus according to claim 11, wherein the computer processor is configured to prevent the robotic unit from performing a stage of a procedure in response to the derived intraocular pressure being greater than a given threshold.

14. The apparatus according to claim 11, wherein the computer processor is configured to prevent the robotic unit from performing a stage of a procedure in response to the derived intraocular pressure being below a given threshold.

15. A method comprising:
direct light toward an eye of a patient;
detect light that is reflected from a surface of the patient's eye, using at least one camera; and
using at least one computer processor:
receiving data from the two or more cameras;
deriving a curvature of the surface of the patient's eye from the received data; and
deriving intraocular pressure of the patient's eye and/or a change in intraocular pressure of the patient's eye from the derived curvature.

16-28. (canceled)

29. Apparatus comprising:
a surgical instrument configured to perform a procedure on a portion of a body of patient;
at least one laser coupled to the surgical instrument and configured to emit a laser beam toward the portion of the patient's body;
one or more cameras configured to image the portion of the patient's body; and
at least one computer processor configured to:
receive data from the one or more cameras,
identify a disposition of at least a portion of the laser beam with respect to the portion of the patient's body based on the received data, and
derive information regarding a disposition of the surgical instrument with respect to the portion of the patient's body from the identified disposition of the laser beam with respect to the portion of the patient's body.

30. The apparatus according to claim 29, wherein the computer processor is configured to move the surgical instrument in response to the derived information regarding the disposition of the surgical instrument with respect to the portion of the patient's body.

31. The apparatus according to claim 29, wherein the computer processor is configured to actuate the surgical instrument to perform a function in response to the derived information regarding the disposition of the surgical instrument with respect to the portion of the patient's body.

32. The apparatus according to claim 29, wherein the computer processor is configured to receive data from a single camera of the one or more cameras and wherein the computer processor is configured to identify a disposition of at least the portion of the laser beam with respect to the portion of the patient's body based on the received data by identifying a point at which the laser beam intersects with a surface of the patient's body.

33. The apparatus according to claim 29, wherein the computer processor is configured to receive data from a stereoscopic camera rig comprising two or more cameras and wherein the computer processor is configured to identify a disposition of at least the portion of the laser beam with respect to the portion of the patient's body based on the received data by triangulating the data from the two or more cameras in order to derive the disposition of the laser beam.

34. The apparatus according to claim 29, wherein the at least one laser comprises two or more lasers configured to emit respective laser beams, with each of the lasers having a respective, different orientation with respect to the surgical instrument, and wherein the computer processor triangulates the laser beams to derive information regarding the disposition of the surgical instrument with respect to the portion of the patient's body.

35. The apparatus according to claim 29, wherein the computer processor is configured to:
receive images of the surgical instrument and the portion of the patient's body from the one or more camera;
identify the surgical instrument and the portion of the patient's body within the images; and
thereby derive an initial derived disposition of the surgical instrument relative to the portion of the patient's body.

36. The apparatus according to claim 35, wherein the computer processor is configured to validate the initial derived disposition of the surgical instrument relative to the portion of the patient's body using the identified disposition of the laser beam with respect to the portion of the patient's body.

37. The apparatus according to claim 35, wherein the computer processor is configured to refine the initial derived disposition of the surgical instrument relative to the portion of the patient's body using the identified disposition of the portion of the laser beam with respect to the portion of the patient's body.

38. The apparatus according to claim 35, wherein, over a course of a procedure, the computer processor is configured to periodically derive an initial derived disposition of the surgical instrument relative to the portion of the patient's body and to refine the initial derived disposition of the surgical instrument relative to the portion of the patient's body using the identified disposition of the portion of the laser beam with respect to the portion of the patient's body.

39. The apparatus according to claim 29, wherein the surgical instrument comprises a tool mounted in an end effector of a robotic arm.

40-41. (canceled)

42. The apparatus according to claim 39, wherein the robotic unit is configured to perform ophthalmic surgery on the patient.

43. The apparatus according to claim 29, wherein the laser is configured to generate a pattern of light, flashes of light, and/or different colors of light in order to convey additional information to the computer processor.

44. The apparatus according to claim 43, wherein:
the surgical instrument comprises a tool mounted in an end effector of a robotic arm,
the laser is coupled to the end effector, and
the computer processor is configured to derive which tool is currently mounted on the end effector, based on the color, the length of the flashes, and/or the pattern of laser light.

45. A method comprising:
performing a procedure on a portion of a body of patient using a surgical instrument;
driving at least one laser coupled to the surgical instrument to emit a laser beam toward the portion of the patient's body;
driving one or more cameras configured to image the portion of the patient's body; and
driving at least one computer processor to:
receive data from the one or more cameras,
identify a disposition of at least a portion of the laser beam with respect to the portion of the patient's body based on the received data, and
derive information regarding a disposition of the surgical instrument with respect to the portion of the patient's body from the identified disposition of the laser beam with respect to the portion of the patient's body.

46-62. (canceled)
